(19) United States
(12) Patent Application Publication
(10) Pub. No.: US 2006/0257968 A1
(43) Pub. Date: Nov. 16, 2006

(54) Portable device for detection of microorganisms

(75) Inventors: Richard Van Duyne, Wilmette, IL (US); Xiaoyu Zhang, Evanston, IL (US); Mathew Young, Evanston, IL (US); Olga Lyandres, Chicago, IL (US)

Correspondence Address: David A. Casimir;Medlin & Carroll, LLP, Suite 350, 101 Howard Street, San Francisco, CA, 94105, US

(73) Assignee: Northwestern University, Evanston, IL (US)

(21) Appl. No.: 11/401,203

(22) Filed: Apr. 10, 2006

Related U.S. Application Data
(60) Provisional application No. 60/669,727, filed on Apr. 8, 2005.

(51) Int. Cl. C12Q 1/04; C12M 1/34

(52) U.S. Cl. 435/34; 977/900; 435/287.1

(57) ABSTRACT

The present invention relates to compositions and methods for detecting microorganisms (e.g., anthrax). In particular, the present invention provides portable, surface-enhanced Raman biosensors, and associated substrates, and methods of using the same, for use in rapidly detecting and identifying microorganisms (e.g., anthrax).

The present invention claims priority to U.S. Provisional Patent Application Ser. No. 60/669,727, filed Apr. 8, 2005, herein incorporated by reference in its entirety.

This invention was funded, in part, under National Science Foundation grant DMR-0076097, and the Air Force Office of Scientific Research MURI program grant F49620-02-1-0381. The government may have certain rights in the invention.

### FIELD OF THE INVENTION

The present invention relates to compositions and methods for detecting microorganisms (e.g., anthrax). In particular, the present invention provides portable, surface-enhanced Raman biosensors, and associated substrates, and methods of using the same, for use in rapidly detecting and identifying microorganisms (e.g., anthrax).

### BACKGROUND OF THE INVENTION

The rapid and accurate identification of bioagents is a vital task for first-responders in order to facilitate timely and appropriate actions in the event of a biological attack. Bacillus anthracis, a spore-forming bacterium and a dangerous pathogen for the disease anthrax, is an important example. B. anthracis bacteria exist in two different forms: rod-shaped organisms and spores. Rod-shaped organisms grow and divide in a nutrient rich environment. When the food supply is depleted, the organisms turn into spores that can survive for decades. Structurally, a spore consists of a central core cell surrounded by various protective layers. Calcium dipicolinate (CaDPA) exists in these protective layers and accounts for ˜10% of the spore's dry weight¹; therefore, it is a useful biomarker for bacillus spores.²

Among the potential biological warfare agent candidates, B. anthracis spores are of particular concern. First, they are highly resistant to environmental stress and are relatively easily produced into weapon-grade material outside the laboratory. Second, anthrax is an infectious disease, requiring medical attention within 24-48 h of initial inhalation of more than 10⁴ B. anthracis spores.³ However, the diagnosis of anthrax is not immediate because it takes 1-60 days for anthrax symptoms to appear in humans.⁴ Therefore, the rapid detection of B. anthracis spores in the environment prior to infection is an extremely important goal for human safety.

Thus, a great need exists for a rapid and sensitive detection protocol suitable for use by first responders to detect dangerous microorganims (e.g., anthrax) that may be used in a biological attack. Furthermore, such materials and methods should be portable for use in the field at potential sites of exposure and capable of providing results on site in short order.

### SUMMARY OF THE INVENTION

The present invention relates to compositions and methods for detecting microorganisms (e.g., anthrax). In particular, the present invention provides portable, surface-enhanced Raman biosensors, and associated substrates, and methods of using the same, for use in rapidly detecting and identifying microorganisms (e.g., anthrax).

A rapid method and portable device suitable for use by first-responders to detect pathogenic bacteria, for example anthrax spores, using a low-cost, battery-powered, portable Raman spectrometer has been developed. The methods and devices of the present invention find use in the detection of a number of different analytes in a variety of different sample types. Preferred embodiments of the present invention find use for the detection of pathogens or components of pathogens. For example, Bacillus subtilis spores, harmless simulants for Bacillus anthracis, were studied using surface-enhanced Raman spectroscopy (SERS) on silver film over nanosphere (AgFON) substrates. AgFON substrates are used for their mechanical robustness, SERS signal stability, and large SERS enhancement factors. These characteristics are important in the development of sensing technology, where reproducibility results with low sample-to-sample variation are of important. Calcium dipicolinate (CaDPA), a biomarker for bacillus spores, was efficiently extracted by sonication in nitric acid and rapidly detected by SERS. The presence of nitric acid from the digestion process additionally provides a convenient internal standard. Improved binding efficiency of the CaDPA adsorption can be further accomplished by using atomic layer deposition (ALD) to coat the AgFON surface with a layer of alumina. The ALD technique permits the precise deposition of materials at Angstrom thicknesses. The alumina layer not only leads to an improvement in Kₐ and more effective measurement, but also helps protect the underlying noble metal surface.

AgFON surfaces optimized for 750 nm laser excitation have been fabricated and characterized by UV-vis diffuse reflectance spectroscopy and SERS. The SERS signal from extracted CaDPA was measured over the spore concentration range of 10⁻¹⁴-10⁻¹² M to determine the saturation binding capacity of the AgFON surface and to calculate the adsorption constant (Kspore=1.7×10¹³ M⁻¹). An 11 minute procedure is capable of achieving a limit of detection (LOD) of approximately 2.6× spores, below the anthrax infectious dose of 10⁴ spores. The data presented herein demonstrate that the shelf life of prefabricated AgFON substrates can be as long as 40 days prior to use. The sensing capabilities of the present invention can be successfully incorporated into a field-portable instrument. Using this technology, 10⁴ bacillus spores were detected with a 5 second data acquisition period on a one-month-old AgFON substrate. The speed and sensitivity of this SERS sensor indicate that this technology is useful for the field analysis of potentially harmful environmental samples.

One embodiment of the present invention is a portable device comprising a plurality of nanobiosensors configured for SERS detection of microorganisms. In some embodiments, the microorganisms are pathogenic bacteria. In some embodiments, the pathogenic bacteria can form spores. In some embodiments, the pathogenic bacteria are from the Bacillus genus, for example B. anthracis. In some embodiments, the portable device is capable of detecting less than 1 microorganismal spores. Preferably, the portable device of the present intention is capable of detecting 2.6×10³ spores of a microorganism. In some embodiments, the device of the present invention is capable of detecting more than 2.6×10³ spores of a microorganism. In some embodiments, the portable device is hand held. In some embodiments, the portable device further comprises a laser excitation power of approximately 50 mW, although the present invention is not so limited.

One embodiment of the present invention comprises a portable device wherein the nanobiosensors of the device comprises nanosphere substrates. In some embodiments, the nanosphere substrates are coated with a noble metal. In some embodiments, the noble metal used to coat the nanosphere substrates is silver. In some embodiments, the silver coated nanosphere substrates are further coated with alumina. In some embodiments, the nanosphere substrates are optimized for 750 nm laser excitation.

One embodiment of the present invention comprises a method of detecting the presence or absence of a microorganism in a sample, comprising: contacting a sample suspected of containing a microorganism with a portable device comprising a plurality of nanobiosensors configured for SERS and detecting the presence or absence of the suspected microorganism by detecting a signal generated from said portable device. In some embodiments, the microorganism being tested for is a pathogenic bacterium. In some embodiments, the pathogenic bacterium is a spore forming bacteria, such as B. anthracis.

### DESCRIPTION OF THE DRAWINGS

FIG. 1. UV-vis diffuse reflectance spectra of different AgFON substrates in air. (A) D=390 nm, dm=200 nm; (B) D=510 nm, dm=200 nm; and (C) D=600 nm, dm=200 nm.

FIG. 2. SERS spectra of 20 μL, 1 mM benzenethiol in ethanol on different AgFON substrates. (A) D=390 nm, dm=200 nm; (B) D=510 nm, dm=200 nm; (C) D=600 nm, dm=200 nm; and (D) D=720 nm, dm=200 nm. The inset shows the variation of the benzenethiol SERS intensity ratio (I1003/I1003,max) with sphere sizes. I1003,max is taken from spectrum 2C. For all spectra, λex=750 nm, Pex=3 mW, acquisition time=1 min.

FIG. 3. (A) SERS spectrum of 3.1×10⁻¹³ M spore suspension (3.7×10⁴ spores in 0.2 μL, 0.02 M HNO₃) on a AgFON substrate. (B) SERS spectrum of 5.0×10⁻⁴ M CaDPA. (C) SERS spectrum of 0.2 μL of 0.02 M HNO₃; λex=750 nm, Pex=50 mW, acquisition time=1 min, D=600 nm, dm=200 nm.

FIG. 4. SERS spectra demonstrate the long-term stability of AgFON substrates, monitored for 1-40 days. SERS spectra of 4.7×10⁻¹⁴ M spore suspension (5.6×10³ spores in 0.2 μL, 0.02 M HNO₃) on AgFON substrates. (A) A 1 day old AgFON, (B) a 15 day old AgFON, and (C) a 40 day old AgFON. The inset shows the intensity ratio (I1020/I1050) variation with time; λex=750 nm, Pex=50 mW, acquisition time=1 min, D=510 nm, and dm=200 nm.

FIG. 5. (A) Adsorption isotherm for B. subtilis spore suspension onto an AgFON substrate. I1020 was taken from SERS spectra that correspond to varying spore concentrations in 0.2 μL of 0.02 M HNO₃ on AgFON substrates; λex=750 nm, Pex=50 mW, acquisition time=1 min, D=600 nm, and dm=200 nm. A Langmuir curve was generated using eq 1 with Kspore=1.3_{—}1013 M−1. The inset shows the linear range that is used to determine the LOD. Each data point represents the average value from three SERS spectra. Error bars show the standard deviations. (B) Adsorption data fit with the linear form of the Langmuir model (eq 2). The slope and intercept values are used to calculate the adsorption constant.

FIG. 6. SERS spectrum of 2.1×10⁻¹⁴ M spore suspension (2.6×10³ spores in 0.2 μL, 0.02 M HNO₃) on AgFON; λex=750 nm, Pex=50 mW, acquisition time=1 min, D=600 nm, and dm=200 nm.

FIG. 7. (A) Adsorption isotherm for CaDPA suspension onto a AgFON substrate. I1020 was taken from SERS spectra that correspond to varying CaDPA concentrations in 0.2 μL of 0.02 M HNO₃ on AgFON substrates; λex=750 nm, Pex=50 mW, acquisition time=1 min, D=600 nm, and dm=200 nm. A Langmuir curve was generated using eq 1 with KCaDPA) 9.5×10³ M−1. The inset shows the linear range that is used to determine the LOD. Each data point represents the average value from three SERS spectra. Error bars show the standard deviations. (B) Adsorption data of CaDPA fit with the linear form of the Langmuir model (eq 2). The slope and intercept values are used to calculate the adsorption constant.

FIG. 8. SERS spectra obtained by the portable Raman spectrometer. (A) SERS spectrum of 8.3×10⁻¹⁴ M spore suspension (1.0×10⁴ spores in 0.2 μL, 0.02 M HNO₃) on 30 day old AgFON. (B) SERS spectrum of 10⁻⁴ M CaDPA in 0.2 μL of 0.02 M HNO3 on 30 day old AgFON substrate; λex=785 nm, Pex=35 mW, acquisition time=5 s, resolution=15 cm−1, D=600 nm, and dm=200 nm.

### DEFINITIONS

As used herein, the term “nanobiosensors configured for surface enhanced Raman spectroscopy detection of an analyte” refers to any small sensor configured to fit within a hand-held device that is specific for detection of one or more analytes, and is capable of having an altered surface enhanced Raman signal in the presence of the specific analyte(s). In preferred embodiments, the nanobiosensors comprise components for specifically, but reversibly, interacting with the specific analyte.

As used herein, the term “surface bound reversibly-binding receptor” refers to a receptor bound to the surface of a nanobiosensor of the present invention that binds reversibly to a specific analyte. In preferred embodiments, the interaction of the receptor and the analyte lasts long enough for detection of the analyte by the sensor.

As used herein, the term “self-assembled monolayer” refers to a material that forms single layer or multilayers of molecules on the surface of a nanobiosensor.

As used herein, the term “nanowell” refers to a solid surface comprising wells for immobilizing the nanobiosensors of the present invention. In preferred embodiments, the nanowells are made of an inert material and are large enough to hold a plurality of nanobiosensors.

As used herein, the term “analyte” refers to any molecule or atom or molecular complex suitable for detection by the nanobiosensors of the present invention. Exemplary analytes include, but are not limited to, various biomolecules (e.g., proteins, nucleic acids, lipids, etc.), pathogens, glucose, ascorbate, lactic acid, urea, pesticides, chemical warfare agents, pollutants, and explosives.

As used herein, the term “a device configured for the detection of surface enhanced Raman scattering signal from said nanobisoensors” refers to any device suitable for detection of a signal from the nanobiosensors of the present invention. In some embodiments, the device includes delivery and collection optics, a laser source, a notch filter, and detector.

As used herein, the term “spectrum” refers to the distribution of electromagnetic energies arranged in order of wavelength.

As used the term “visible spectrum” refers to light radiation that contains wavelengths from approximately 360 nm to approximately 800 nm.

As used herein, the term “ultraviolet spectrum” refers to radiation with wavelengths less than that of visible light (i.e., less than approximately 360 nm) but greater than that of X-rays (i.e., greater than approximately 0.1 nm).

As used herein, the term “infrared spectrum” refers to radiation with wavelengths of greater than 800 nm.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as bacteria, surface matter, soil, water, crystals and industrial samples. Such examples are not however to be construed as limiting the sample types applicable to the present invention.

As used herein, the term “medium” refers to the fluid environment of an analyte of interest. In some embodiments, the medium refers to a bodily fluid. The bodily fluid may be, for example, blood, plasma, serum, cerebrospinal fluid, vitreous or aqueous humor, urine, extracellular fluid, or interstitial fluid. In some embodiments, the medium is an in vivo medium. In other embodiments, the medium is an ex vivo or in vitro medium, for example, a fluid sample taken from a subject. In other embodiments, the medium is a liquid that is found on the surface of an object or structure, wherein which a bacteria or other biological organism (e.g., pathogen) is present or expected to be present.

As used herein, the term “pathogen” refers or an agent that can cause disease in other organisms or in humans, animals and plants (eg, bacteria, viruses, or parasites). Examples of pathogens of the present invention include microorganisms capable of causing damage or harm to a living organism. For example, B. anthracis is a pathogen capable of causing anthrax in humans and non-human animals, and is considered a biological weapon.

### DETAILED DESCRIPTION OF THE INVENTION

In the last two decades, various biological and chemical techniques have been developed to detect bacillus spores. Two important biological methods are the polymerase chain reaction (PCR)⁵⁻⁷ and immunoassays.^{8,9} PCR, a primer-mediated enzymatic DNA amplification method, requires expensive reagents, molecular fluorophores, and considerable sample processing prior to analysis. The limit of detection (LOD) based on PCR detection of bacterial pagA gene is ×10³ spores in 3 h.⁷ Immunoassays, which rely on the interaction between antibodies and B. anthracis cell surface antigens, can detect 10⁵ spores in 15 min.⁹ However, in immunoassays, it is necessary to employ specific antibodies for the desired agents and to individually adjust the mobile-phase conditions for their capture, elution, and separation.

Recently, relatively rapid chemical methods for the detection of bacillus spores have been developed. For instance, photoluminescence detection based on the formation of terbium (Tb(III)) dipicolinate was found to have a LOD of 103 B. subtilis colony-forming-units mL⁻¹ in 5-7 min.¹⁰ This method compares the enhanced luminescence of the terbium dipicolinate complex to Tb(III) alone. However, an increase in the luminescence intensity can also occur by the complexation of Tb(III) with aromatic compounds other than dipicolinic acid.¹¹ Due to the frequency of false positives and its limited ability for target analyte identification, alternative approaches with improved selectivity would be a welcome addition to the arsenal of anthrax detection methods.

Compared to photoluminescence, vibrational spectroscopy possesses highly specific chemical information content and, therefore, is capable of uniquely identifying target analytes. Both Fourier transform infrared (FT-IR)¹² and Raman^{13,14} spectroscopies have demonstrated the ability to discriminate among different bacterial spores. However, the implementation of nearinfrared (NIR) and mid-infrared spectroscopies has fundamental limitations due to the competitive absorption of water and inherent spectral congestion. In contrast, Raman spectroscopy is well-suited to applications in aqueous environments because of the small Raman scattering cross section of water¹⁵ (See, e.g., U.S. Pat. App. No. 20040180379, herein incorporated by reference in its entirety). Recently, for example, single bacterial spores have been detected using micro-Raman spectroscopy.¹⁴ As a consequence of different individual sporulations, however, the micro-Raman spectra vary significantly from one spore to another. Additionally, the applications that require complex instrumentation used in this approach restrict its applicability in field-portable measurements. The detection of bacillus spores by normal Raman spectroscopy (NRS) has also been demonstrated;¹⁶ however, NRS suffers from low sensitivity, so that long data acquisition times (5-13 min) and high laser powers (400 mW) are required. In comparison, surface-enhanced Raman spectroscopy (SERS) yields more intense Raman signals at much lower laser excitation power. SERS produces very large enhancements in the effective Raman cross section of species spatially confined within the electromagnetic fields generated by excitation of the localized surface plasmon resonance (LSPR) of nanostructured noble metal surfaces.¹⁷ The SERS signals of ensemble-averaged molecules show enhancements up to 8 orders of magnitude over NR signals.¹⁸ Furthermore, the low power required by SERS allows the development of a compact, field-portable detection system.

Calcium dipicolinate (CaDPA) is a component of the bacterial spore case. The process of sporogenesis occurs when the vegetative bacterial cell segregates its contents to form a sporangium. The sporangium content includes what will be the future bacterial spore, also known as an endospore. Upon sporogenesis, the water is completely removed from the endospore by the sporangium, which is then covered by a cortex layer rich in calcium dipicolinate. CaDPA can be a major component of the spore case. In fact, CaDPA can comprise 5-20% of the spore case, with the concentration varying among different genera and species of spore forming bacteria.

Accordingly, the present invention provides compositions and methods for the rapid extraction of CaDPA from B. subtilis spores, simulants for B. anthracis spores, and SERS detection on reproducible,¹⁹ stable^{20,21} silver film over nanosphere (AgFON) substrates or similar substrates. However, the present invention is not limited to Bacillus species, as any bacterium that is capable of forming spores which contain CaDPA in the spore case are equally amenable to the present invention. Indeed, those skilled in the art will recognize that a number of pathogenic bacteria form spores. For example, additional spore forming pathogens include, but are not limited to, B. cereus (food poisoning) Clostridium perfringens (gangrene), and C. botulinum (food poisoning). Any of the pathogenic bacteria listed herein could be potentially utilized as a biological weapon. It is contemplated that CaDPA in the spore case of the aforementioned pathogens can be detected using the device and methods of the present invention. Additional examples of spore forming microorganisms can be found in Bacterial Spore Formers: Probiotics & Emerging Applications, E. Ricca et al., Eds, Horizon Scientific Press, p. 244, incorporated herein in its entirety.

When the localized surface plasmon resonance (LSPR) maximum of a AgFON substrate closely matches the laser excitation wavelength, the maximum SERS signal intensity results.^{18,22} In some embodiments, AgFON surfaces are fabricated using 600 nm spheres in order to optimize SERS intensity for 750 nm laser excitation. In some embodiments, the present invention provides detection of a LOD of ˜2.6×10³ spores with a data acquisition period of 1 min and a laser power of 50 mW. In other embodiments, the present invention detects LOD of less than 2.6×10³ spores. In still further embodiments, the present invention detects a LOD of greater than 2.6×10³ spores.

Previously published SERS studies of anthrax detection via the CaDPA biomarker were 200 times less sensitive and required 3 times more laser power²³ than that of the present invention. Similarly, previous published NRS studies were 200,000 times less sensitive and required 8 times more laser power.¹⁶ than the compositions and methods of the present invention. The present invention also provides AgFON substrates that provide stable SERS spectra for at least 40 days. The present invention further provides a portable SERS device that produces a SERS spectrum from 10⁴ spores in 5 s using a 1-month-old prefabricated AgFON. Thus, the present invention provides the first compact vibrational spectrometer for the detection of bacillus spores.

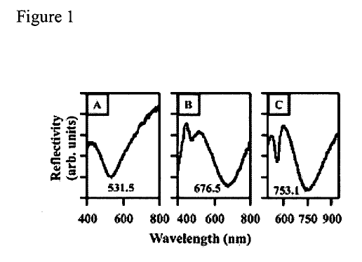

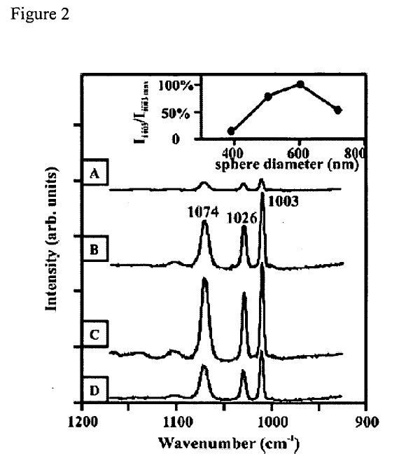

The present invention contemplates the use of SERS for rapid detection of the anthrax biomarker, CaDPA, using a low-cost, battery-powered, and portable Raman spectrometer, although other biomarkers may be detected. It is further contemplated that the present invention will detect any microorganism that contains CaDPA in detectable quantities. Typically, such spectrometers use an NIR diode laser as the excitation source. One popular diode laser excitation wavelength is 785 nm. To mimic a 785 nm diode laser, the present invention, in some embodiments, uses a CW Ti:Sa laser tuned to 750 nm as the laser excitation source. In some embodiments, the NIR excitation reduces the native fluorescence background from microorganisms. Previously, an important correlation between nanoparticle structure, as reported by the spectral position of the LSPR relative to the laser excitation wavelength, and the SERS intensity was demonstrated.^{18,22} The maximum SERS intensity is obtained from a AgFON surface when the laser excitation wavelength coincides with the LSPR maximum. Since AgFONs are not optically transparent, the reflectivity minimum was used to locate the LSPR maximum. In some embodiments, AgFON substrates for SERS measurements using 750 nm laser excitation are optimized by first measuring the dependence of the LSPR spectral position on nanosphere diameter. FIG. 1 shows the UV-vis diffuse reflectance spectra of AgFON substrates with nanospheres having diameters of 390, 510, and 600 nm. In some embodiments, a AgFON sample was also fabricated using 720 nm diameter spheres. In FIG. 1, the reflectance spectrum of AgFON substrate C (nanosphere diameter, D=600 nm, and mass thickness of Ag film, dm=200 nm) shows a reflectivity minimum near 753 nm, attributable to the excitation of the LSPR of the silver film. This substrate is expected to show the largest intensity for 750 nm laser excitation. To further confirm this expectation, SERS spectra of 1 mM benzenethiol in 20 μL of ethanol on the AgFON substrates with D=390, 510, 600, and 720 nm (FIG. 2) were measured. The largest SERS enhancement of benzenethiol was, in fact, observed from the AgFON with D=600 nm and dm=200 nm (FIG. 2C). In some embodiments, this AgFON substrate was chosen as optimal for the bacillus spore detection experiments.

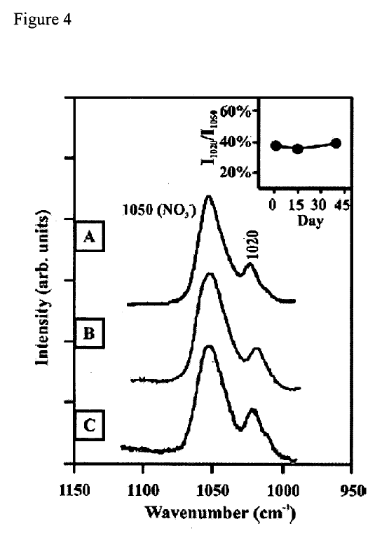

It is contemplated that an ideal detection system would run unattended for long periods of time, require infrequent maintenance, and operate at low cost. Previous work has demonstrated that bare AgFON surfaces display extremely stable SERS activity when challenged by negative potentials in electrochemical experiments²⁰ and high temperatures in ultrahigh vacuum experiments.²⁸ The present invention provides information regarding the temporal stability of AgFON substrates studied over a period of 40 days. SERS spectra of 4.7×10⁻¹⁴ M spores (5.6×10³ spores in 0.2 μL, 0.02 M HNO3), well below the anthrax infectious dose of 10⁴ spores, were captured on AgFON substrates of different ages (FIG. 4). The intensity ratios between the strongest CaDPA peak at 1020 cm−1 and the NO3-peak at 1050 cm−1 (I1020/I1050) were measured to quantitatively compare the AgFON substrates of different ages (shown in FIG. 4 inset). Both the CaDPA spectral band positions and intensity patterns remained constant over the course of 40 days, indicating the long-term stability of the AgFON as SERS substrates for potential field-sensing applications.

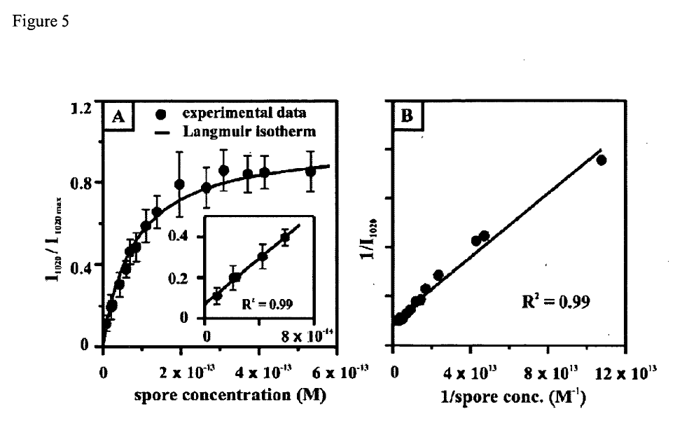

The quantitative relationship between SERS signal intensity and spore concentration is demonstrated in FIG. 5A. Each data point represents the average intensity at 1020 cm−1 from three samples, with the standard deviation shown by the error bars. At low spore concentrations, the peak intensity increases linearly with concentration (FIG. 5A inset). At higher spore concentrations, the response saturates as the adsorption sites on the AgFON substrate become fully occupied. Saturation occurs when the spore concentrations exceed ˜2.0×10⁻¹³ M (2.4×10⁴ spores in 0.2 μL, 0.02 M HNO3).

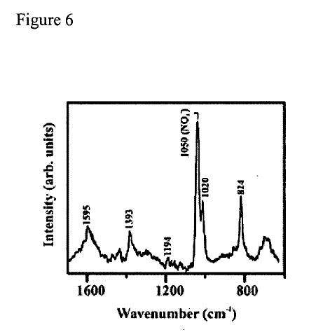

It is preferential that a SERS-based detection system be capable of detecting less than the life-threatening dose of a pathogen in real or near real time. In the present invention, the LOD is the concentration of spores for which the strongest SERS signal of CaDPA at 1020 cm−1 is equal to 3 times the background SERS signal within a 1 min acquisition period. The background signal refers to the SERS intensity from a sample with a spore concentration equal to 0, which is calculated to be the intercept of the low concentration end of the spore adsorption isotherm (FIG. 5A). In some embodiments, lower detection limits are achieved using longer acquisition times. In some embodiments, these aforementioned parameters are used for high throughput, real-time, and on-site analysis of potentially harmful species. For example, the LOD for B. subtilis spores, evaluated by extrapolation of the linear concentration range of the adsorption isotherms (FIG. 5A inset), is found to be 2.1×10⁻¹⁴ M (2.6×10³ spores in 0.2 μL, 0.02 M HNO₃). Furthermore, when a similar spore concentration (2.1×10⁻¹⁴ M, 2.6×10³ spores in 0.2 μL, 0.02 M HNO₃) is deposited onto a AgFON surface, a 1 min acquisition yields a SERS spectrum that clearly displays the spore Raman features at 1595, 1393, 1020, and 824 cm−1 (FIG. 6). These data demonstrate that the SERS LOD is below the anthrax infectious dose of 10⁴ spores.

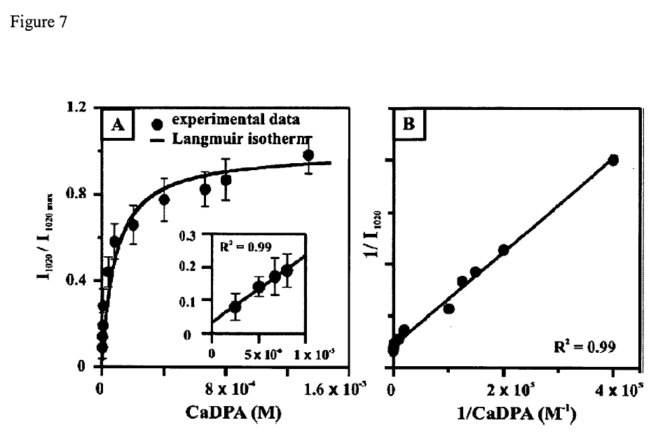

To determine the adsorption capacity of extracted CaDPA on a AgFON, the Langmuir adsorption isotherm was used to fit the data:^{29,30}:

$$\begin{matrix}θ = \frac{{I}_{1020}}{{I}_{1020 , max}} = \frac{{K}_{spore} \times [ spore ]}{1 + {K}_{spore} \times [ spore ]} & ( 1 ) \\ \frac{1}{{I}_{1020}} = \frac{1}{{K}_{spore} \times {I}_{1020 , max}} \times \frac{1}{[ spore ]} + \frac{1}{{I}_{1020 , max}} & ( 2 )\end{matrix}$$

where θ is the coverage of CaDPA on the AgFON; I1020, max is the maximum SERS signal intensity at 1020 cm−1 when all the SERS active sites on AgFON are occupied by CaDPA; [spore] is the concentration of spores (M), and Kspore is the adsorption constant of CaDPA extracted from spores on AgFON (M−1). From eq 2, Kspore is calculated from the ratio between the intercept and the slope. Slope and intercept analyses of the linear fit (FIG. 5B) lead to the value of the adsorption constant, Kspore) 1.7×10¹³ M−1. Parallel studies of SERS intensities versus CaDPA concentrations indicate that the LOD is 3.1×10⁻⁶ M in 0.2 μL, 0.02 M HNO₃ (FIG. 7A inset), and the adsorption constant for CaDPA, KCaDPA, is 9.0×10³ M-1. The ratio between Kspore and KCaDPA represents the extracted amount of CaDPA. Accordingly, it can be estimated that approximately 1.9×10⁹ mol DPA is extracted from 1 mol spores, which corresponds to 3.0% of spore weight. Previous research found that B. subtilis spores contain approximately 8.9% DPA by weight.¹ Therefore, the DPA extraction efficiency of 10 min sonication in 0.02 M HNO₃ is ˜34%.

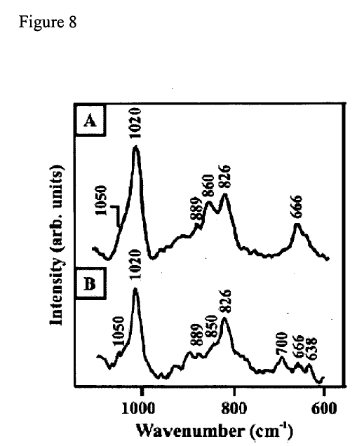

The present invention provides a SERS as a field-portable screening tool by using a compact Raman spectrometer. It is contemplated that the field portable tool be a hand held device, or a portable device capable of being utilized in any location (e.g., outside in the environmental, inside in a building, etc.). Many field-sensing applications require the portability and flexibility not available from conventional laboratory scale spectroscopic equipment. As a first step in this direction, the Raman spectrum from 10⁴ B. subtilis spores dosed onto a 1 month old AgFON substrate was readily acquired using a commercially available portable Raman instrument. A high S/N spectrum was achieved within 5 seconds (FIG. 8A). The SERS peak positions and intensity pattern for the spore sample were similar to those of CaDPA recorded utilizing the same device (FIG. 8B). Thus, the present invention provides the first example of using a compact, portable Raman spectrometer for the detection of bacillus spores. The portability and ease of use of this type of device with the molecular specificity and spectral sensitivity inherent to SERS open a range of possibilities for detecting bioagents and other chemical threats in an outdoor, field environment as well as in buildings and small, internal and external spaces.

Accordingly, the present invention provides realtime detection of anthrax spores using SERS. In some embodiments, AgFON surfaces (D=600 nm and dm=200 mm) were determined to be SERS intensity-optimized substrates for 750 nm laser excitation. CaDPA was rapidly extracted from B. subtilis spores using a 10 min sonication in 0.02 M HNO₃, with an extraction efficiency of ˜34%. In some embodiments, the peaks associated with CaDPA dominate the SERS spectrum of spores. In some embodiments, the strongest peak of CaDPA at 1020 cm−1 is used to measure SERS intensity versus spore concentration profiles that yield an adsorption constant, Kspore=1.7×10¹³ M−1. On the basis of the linear portion of the response curve, the LOD of B. subtilis spores was estimated to be 2.1×10⁻¹⁴ M (2.6×10³ spores in 0.2 μL, 0.02 M HNO3) for a 1 min data acquisition period. Furthermore, in some embodiments, the detection level is well below the anthrax infectious dose of 1 spores, and can easily be measured within this acquisition times noted above.

The following examples serve to illustrate certain preferred embodiments and aspects of the present invention and are not to be construed as limiting the scope thereof.

Materials and Methods

All of the chemicals used were of reagent grade or better. Ag (99.99%) was purchased from D. F. Goldsmith (Evanston, Ill.). Glass substrates were 18 mm diameter, No. 2 cover slips from Fisher Scientific (Pittsburgh, Pa.). Pretreatment of substrates required H2SO4, H2O2, and NH4OH, all of which were purchased from Fisher Scientific (Fairlawn, N.J.). Surfactant-free white carboxyl-functionalized polystyrene latex nanospheres with diameters of 390, 510, 600, and 720 nm were obtained from Duke Scientific Corporation (Palo Alto, Calif.) and Interfacial Dynamics Corporation (Portland, Oreg.). Tungsten vapor deposition boats were purchased from R. D. Mathis (Long Beach, Calif.). Nitric acid 70% (Fisher Scientific), dipicolinic acid (2,6-pyridinedicarboxylic acid, DPA), calcium hydroxide, and benzenethiol (Aldrich Chemical Co., Milwaukee, Wis.) were used as purchased. Water (18.2 MΩ/cm) was obtained from an ultrafilter system (Milli-Q, Millipore, Marlborough, Mass.). Calcium dipicolinate (CaDPA) was prepared from DPA and calcium hydroxide according to the method of Beiley and co-workers.

The SERS apparatus comprising a battery-powered Raman spectrometer (model Inspector Raman, diode laser excitation wavelength λex 785 nm) was purchased from DeltaNu (Laramie, Wyo.) and was used to demonstrate the feasibility of a field-portable device for spore detection. The remaining data were acquired using a macro-Raman system. This system comprises an interference filter (Coherent, Santa Clara, Calif.), a 1 in. holographic edge filter (Physical Optics Corporation, Torrance, Calif.), a single-grating monochromator with the entrance slit set at 100 /m (model VM-505, Acton Research Corporation, Acton, Mass.), a liquid-N-2-cooled CCD detector (Model Spec-10:400B, Roper Scientific, Trenton, N.J.), and a data acquisition system (Photometrics, Tucson, Ariz.). A titanium-sapphire laser (CW Ti:Sa, Model 3900, Spectra Physics, Mountain View, Calif.) pumped by a solid-state diode laser (Model Millenia Vs, Spectra Physics) was used to generate λex of 750 nm. All of the measurements were performed in ambient conditions.

### EXAMPLE 1

### Microbial Culture

B. subtilis was purchased from the American Type Culture Collection (Manassas, Va.). Spore cultures were cultivated by spreading the vegetative cells on sterile nutrient agar plates (Fisher Scientific), followed by incubating at 30° C. for 6 days. The cultures were washed from the plates using sterile water and centrifuged at 12 000 g for 10 min. The centrifuging procedure was repeated five times. The lyophilized spores were kept at 2-4° C. prior to use. Approximately 1 g of sample was determined to contain 5.6×10¹⁰ spores by optical microscopic measurements (data not shown). The spore suspension was made by dissolving spores in 0.02 M HNO₃ solution and by sonicating for 10 min.

### EXAMPLE 2

### Extraction of CaDPA from Spores

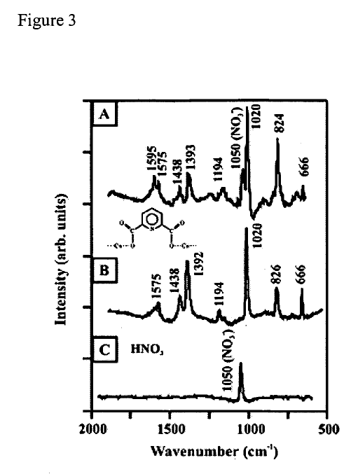

CaDPA was extracted from spores by sonicating in 0.02 M HNO₃ solution for 10 min. This concentration of the HNO₃ solution was selected because of its capability of extraction and its benign effect on the AgFON SERS activity. To test the efficiency of this extraction method, a 3.1×10⁻¹³ M spore suspension (3.7×10⁴ spores in 0.2 μL, 0.02 M HNO₃) was deposited onto an AgFON substrate (D=600 nm, dm=200 nm). The sample was allowed to evaporate for less than 1 min. A high signal to-noise ratio (S/N) SERS spectrum was obtained in 1 min (FIG. 3A). For comparison, a parallel SERS experiment was conducted using 5.0×10⁻⁴ M CaDPA (FIG. 3B). The SERS spectrum of B. subtilis spores is dominated by bands associated with CaDPA, in agreement with the previous Raman studies on bacillus spores.^{16,23} The SERS spectra in FIG. 3, however, display noticeable differences at 1595 cm−1, which are from the acid form of dipicolinate.²⁶ The peak at 1050 cm−1 in FIG. 3A is from the symmetrical stretching vibration of NO³⁻.²⁷ The NO³⁻ peak can be used as an internal standard to reduce the sample-to-sample deviations if desired.

### EXAMPLE 3

### AgFON Substrate Fabrication

Glass substrates were pretreated in two steps; 1) Piranha etch, 3:1H₂SO₄/30% H₂O₂ at 80° C. for 1 h, was used to clean the substrate, and 2) base treatment, of 5:1:1H₂O/NH₄OH/30% H₂O₂ with sonication for 1 h was used to render the surface hydrophilic. Approximately 2 μL of the nanosphere suspension (4% solids) was drop coated onto each substrate and allowed to dry in ambient conditions. The metal films were deposited in a modified Consolidated Vacuum Corporation vapor deposition system with a base pressure of 10-6 Torr.²⁴ The deposition rates for each film (10 Å/s) were measured using a Leybold Inficon XTM/2 quartz crystal microbalance (QCM) (East Syracuse, N.Y.). AgFON substrates were stored in the dark at room temperature prior to use.

### EXAMPLE 4

### UV-Vis Diffuse Reflectance Spectroscopy

Measurements were carried out using an Ocean Optics (Dunedin, Fla.) SD2000 spectrometer coupled to a reflection probe (Ocean Optics) and a halogen lamp (Model F-O-Lite H, World Precision Instruments, Sarasota, Fla.). The reflection probe consists of a tight bundle of 13 optical fibers (12 illumination fibers around a collection fiber) with a usable wavelength range of 400-900 nm. All reflectance spectra were collected against a mirrorlike Ag film over glass substrate as a reference.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

### REFERENCES

(1) Bailey, G. F.; Karp, S.; Sacks, L. E. J. Bacteriol. 1965, 89, 984-987.
(2) Goodacre, R.; Shann, B.; Gilbert, R. J.; Timmins, E. M.; McGovern, A. C.; Alsberg, B. K.; Kell, D. B.; Logan, N. A. Anal. Chem. 2000, 72, 119-127.
(3) Walt, D. R.; Franz, D. R. Anal. Chem. 2000, 72, 738A-746A.
(4) Chin, J. E. Control of Communicable Diseases Manual; American Public Health Association: Washington, D.C., 2000; pp 20-25.
(5) De Wit, M. Y.; Faber, W. R.; Krieg, S. R.; Douglas, J. T.; Lucas, S. B.; Montreewasuwat, N.; Pattyn, S. R.; Hussain, R.; Ponnighaus, J. M.; Hartskeerl, R. A. J. Clin. Microbiol. 1991, 29, 906-910.
(6) Hurtle, W.; Bode, E.; Kulesh, D. A.; Kaplan, R. S.; Garrison, J.; Bridge, D.; House, M.; Frye, M. S.; Loveless, B.; Norwood, D. J. Clin. Microbiol. 2004, 42, 179-185.
(7) Fasanella, A.; Losito, S.; Adone, R.; Ciuchini, F.; Trotta, T.; Altamura, S. A.; Chiocco, D.; Ippolito, G. J. Clin. Microbiol. 2003, 41, 896-899.
(8) Yolken, R. H.; Wee, S. B. J. Clin. Microbiol. 1984, 19, 356-360.
(9) King, D.; Luna, V.; Cannons, A.; Cattani, J.; Amuso, P. J. Clin. Microbiol. 2003, 41, 3454-3455.
(10) Pellegrino, P. M.; Fell, N. F.; Gillespie, J. B. Anal. Chim. Acta 2002, 455, 167.
(11) Beltyukova, S. V.; Poluektov, N. S.; Tochidlovskaya, T. L.; Kucher, A. A. Dokl. Akad. Nauk. 1986, 291, 1392-1395.
(12) Thompson, S. E.; Forster, N. S.; Johnson, T. J.; Valentine, N. B.; Amonette, J. E. Appl. Spectrosc. 2003, 57, 893-899.
(13) Jarvis, M. R.; Goodacre, R. Anal. Chem. 2004, 76, 40-47.
(14) Chan, J. W.; Esposito, A. P.; Talley, C. E.; Hollars, C. W.; Lane, S. M.; Huser, T. Anal. Chem. 2004, 76, 599-603.
(15) McCreery, R. L. Raman Spectroscopy for Chemical Analysis; John Wiley & Sons: New York, 2000; Vol. 157, p 420.
(16) Farquharson, S.; Grigely, L.; Khitrov, V.; Smith, W.; Sperry, J. F.; Fenerty, G. J. Raman Spectrosc. 2004, 35, 82-86.
(17) Schatz, G. C.; Van Duyne, R. P. Electromagnetic Mechanism of Surface-Enhanced Spectroscopy. In Handbook of Vibrational Spectroscopy; Chalmers, J. M., Griffiths, P. R., Eds.; Wiley: New York, 2002; Vol. 1, pp 759-774.
(18) Haynes, C. L.; Van Duyne, R. P. J. Phys. Chem. B 2003, 107, 7426-7433.
(19) Dick, L. A.; McFarland, A. D.; Haynes, C. L.; Van Duyne, R. P. J. Phys. Chem. B 2002, 106, 853-860.
(20) Zhang, X.; Yonzon, C. R.; Van Duyne, R. P. Proc. SPIE-Int. Soc. Opt. Eng. 2003, 5221, 82-91.
(21) Yonzon, C. R.; Haynes, C. L.; Zhang, X.; Walsh, J. T. J.; Van Duyne, R. P. Anal. Chem. 2004, 76, 78-85.
(22) McFarland, A. D. Ph.D. Thesis, Northwestern Universtiy, 2004.
(23) Farquharson, S.; Gift, A. D.; Maksymiuk, P.; Inscore, F. E. Appl. Spectrosc. 2004, 58, 351-354.
(24) Hulteen, J. C.; Van Duyne, R. P. J. Vac. Sci. Technol. A 1995, 13, 1553-1558.
(25) Deltanu Company Home Page; http://www.deltanu.com (accessed July, 2004).
(26) Carmona, P. Spectrochim. Acta; Part A 1980, 36A, 705-712.
(27) Mosier-Boss, P. A.; Lieberman, S. H. Appl. Spectrosc. 2000, 54, 1126-1135.
(28) Litorja, M.; Haynes, C. L.; Haes, A. J.; Jensen, T. R.; Van Duyne, R. P. J. Phys. Chem. B 2001, 105, 6907-6915.
(29) Jung, L. S.; Campbell, C. T. J. Phys. Chem. B 2000, 104, 11168-11178.
(30) Jung, L. S.; Campbell, C. T. Phys. Re. Lett. 2000, 84, 5164-5167.

We claim:

1. A portable device comprising a plurality of nanobiosensors, said nanobiosensors configured for surface enhanced Raman spectroscopy detection of microorganisms.

2. The device of claim 1, wherein said nanobiosensors comprise nanosphere substrates.

3. The device of claim 2, wherein said nanosphere substrates are coated with a noble metal.

4. The device of claim 3, wherein said noble metal coated nanosphere substrates are further coated with alumina.

5. The device of claim 3, wherein said noble metal comprises silver.

6. The device of claim 1, wherein said nanobiosensors are configured for quantitative detection of said microorganisms.

7. The device of claim 6, wherein said level of quantitative detection is 2.0×10³ spores or less of said microorganism.

8. The device of claim 1, wherein said microorganism is a pathogen.

9. The device of claim 8, wherein said pathogen is capable of forming spores.

10. The device of claim 9, wherein said spore forming pathogen is Bacillus anthracis.

11. The device of claim 1, wherein said microorganism is Bacillus anthracis.

12. The device of claim 2, wherein said nanosphere substrates are 500-700 nm in size.

13. The device of claim 12, wherein said nanosphere substrates are optimized for 750 nm laser excitation.

14. The device of claim 1, further comprising a laser excitation power of 50 mW or less.

15. A method for detecting the presence or absence of microorganisms comprising:
a) providing a sample suspected of containing a microorganism,
b) contacting said sample with a portable device comprising a plurality of nanobiosensors configured for surface enhanced Raman spectroscopy, and
c) detecting the presence or absence of said microorganism in said sample by detecting a surface enhanced Raman spectroscopy signal from said portable device.

16. The method of claim 15, wherein said microorganism is a pathogenic bacteria.

17. The method of claim 16, wherein said pathogenic bacteria is capable of forming spores.

18. The method of claim 17, wherein said pathogenic bacteria capable of forming spores is from a group consisting of Bacillus subtilis and Bacillus anthracis.

19. The method of claim 15, wherein said portable device is a hand held device.

20. The method of claim 15, wherein said nanobiosensors comprise nanosphere substrates.

21. The method of claim 20, wherein said nanosphere substrates are coated with a noble metal.

22. The method of claim 21, wherein said noble metal is silver.

23. The method of claim 21, wherein said noble metal coated biosensors are further coated with alumina.

24. The method of claim 15, wherein said detection is less than 10⁴ spores of said microorganism.
